(19) United States
(12) Patent Application Publication
(10) Pub. No.: US 2021/0375404 A1
(43) Pub. Date: Dec. 2, 2021

(54) MEDICAL QUESTION-ANSWERING METHOD, MEDICAL QUESTION-ANSWERING SYSTEM, ELECTRONIC DEVICE, AND COMPUTER READABLE STORAGE MEDIUM

(71) Applicant: BOE TECHNOLOGY GROUP CO., LTD., Beijing (CN)

(72) Inventor: Yulan HU, Beijing (CN)

(21) Appl. No.: 17/282,035

(22) PCT Filed: Jun. 3, 2020
(86) PCT No.: PCT/CN2020/094068

§ 371 Date: Apr. 1, 2021

(30) Foreign Application Priority Data
Jun. 5, 2019 (CN) 201910484808.4

(51) Int. Cl. G16H 10/20; G06F 40/205; G06F 40/247; G06F 40/30

(52) U.S. Cl. CPC G16H 10/20; G06F 40/30; G06F 40/247; G06F 40/205

(57) ABSTRACT

The present disclosure provides a medical question-answering method, including: recognizing an intention of a patient according to a medical consultation sentence input by the patient, extracting at least one substantive term corresponding to disease characteristics from the medical consultation sentence according to the intention of the patient, acquiring a standard term synonymous with each of the at least one substantive term according to a preset synonym mapping table, wherein the synonym mapping table comprises mapping relationships between a plurality of standard terms and corresponding substantive terms, generating a semantic parsing result according to the intention of the patient and the standard term, and outputting a corresponding answer according to the semantic parsing result. The present disclosure further provides a medical question-answering system, an electronic device, and a computer readable storage medium.

### CROSS REFERENCE TO RELATED APPLICATIONS

The present application claims the priority of Chinese patent application No. 201910484808.4, filed on Jun. 5, 2019, the content of which is incorporated herein by reference in its entirety.

### TECHNICAL FIELD

The present disclosure relates to the field of internet technologies, and in particular, to a medical question-answering method, a medical question-answering system, an electronic device, and a computer readable storage medium.

### BACKGROUND

With rapid development of internets, in a health-related medical field, many online disease question-answering (e.g., questioning and answering) websites appear, and may provide constructive disease diagnosis suggestions to a patient in an early stage of a disease. However, a question asked by the patient may have problems such as colloquialization and diversity of description. Therefore, it is necessary to correctly understand and answer the question input by the patient.

### SUMMARY

Embodiments of the present disclosure provide a medical question-answering method, a medical question-answering system, an electronic device, and a non-transitory computer readable storage medium.

A first aspect of the present disclosure provides a medical question-answering method, including:

recognizing an intention of a patient according to a medical consultation sentence input by the patient;

extracting at least one substantive term corresponding to disease characteristics from the medical consultation sentence according to the intention of the patient;

acquiring a standard term synonymous with each of the at least one substantive term according to a preset synonym mapping table, wherein the synonym mapping table includes mapping relationships between a plurality of standard terms and corresponding substantive terms;

generating a semantic parsing result according to the intention of the patient and the standard term; and

outputting a corresponding answer according to the semantic parsing result.

In an embodiment, the recognizing an intention of a patient according to a medical consultation sentence input by the patient includes:

acquiring document subject information of the medical consultation sentence input by the patient;

converting the medical consultation sentence input by the patient from text data into vector data;

acquiring a score of the medical consultation sentence for each of preset intentions according to the document subject information and the vector data corresponding to the medical consultation sentence; and

determining the intention of the patient according to the score of the medical consultation sentence for each of the preset intentions.

In an embodiment, the extracting at least one substantive term corresponding to disease characteristics from the medical consultation sentence according to the intention of the patient includes:

acquiring a semantic slot template corresponding to the intention of the patient, wherein the semantic slot template includes a plurality of semantic slots for representing the disease characteristics; and

extracting substantive terms corresponding to the plurality of semantic slots in the semantic slot template from the medical consultation sentence.

In an embodiment, the extracting substantive terms corresponding to the plurality of semantic slots in the semantic slot template from the medical consultation sentence includes:

performing sequence labeling on the medical consultation sentence by using a sequence labeling model, and acquiring the substantive terms corresponding to the plurality of semantic slots in the semantic slot template according to a sequence labeling result.

In an embodiment, the generating a semantic parsing result according to the intention of the patient and the standard term includes:

filling the standard term corresponding to the medical consultation sentence of the patient into corresponding one of the plurality of semantic slots;

determining whether there is an unfilled semantic slot in a current semantic slot template;

in a case where a result of determination indicates that there is an unfilled semantic slot in the current semantic slot template, generating an inquiry question corresponding to the unfilled semantic slot, and filling the unfilled semantic slot according to an answer sentence input by the patient for the inquiry question, until all of the semantic slots in the current semantic slot template are filled; and

generating the semantic parsing result according to the intention of the patient, each semantic slot, and a filled value of the semantic slot.

In an embodiment, the outputting a corresponding answer according to the semantic parsing result includes:

calculating a matching degree between the semantic parsing result and each sample group in a doctor-patient question-answering knowledge library, wherein each sample group includes a question sample and its corresponding answer sample; and

outputting an answer sample having a maximum matching degree.

In an embodiment, the calculating a matching degree between the semantic parsing result and each sample group in a doctor-patient question-answering knowledge library includes:

calculating a similarity between the semantic parsing result and the question sample and a correlation between the semantic parsing result and the answer sample; and

generating the matching degree according to the similarity, a first weighting coefficient, the correlation, and a second weighting coefficient.

In an embodiment, the disease characteristics includes at least one of an onset symptom, a symptom onset time, a symptom duration, an accompanying symptom, a medical history, a treatment history, and an age of the patient.

In an embodiment, prior to the recognizing an intention of a patient according to a medical consultation sentence input by the patient, the medical question-answering method further includes:

generating a standard term library in which a plurality of standard term samples are stored;

collecting at least one synonym corresponding to each of the plurality of standard term samples;

calculating a similarity between each standard term sample and its corresponding synonym, maintaining each synonym having a similarity greater than a preset value, and removing each synonym having a similarity less than or equal to the preset value; and

generating the synonym mapping table according to each synonym and its corresponding synonym which is maintained currently.

A second aspect of the present disclosure provides a medical question-answering system, including:

an intention recognizer, configured to recognize an intention of a patient according to a medical consultation sentence input by the patient;

a substantive term extractor, configured to extract at least one substantive term corresponding to disease characteristics from the medical consultation sentence according to the intention of the patient;

a standard term acquisition unit, configured to acquire a standard term synonymous with each of the at least one substantive term according to a preset synonym mapping table, wherein the synonym mapping table includes mapping relationships between a plurality of standard terms and corresponding substantive terms;

an parser, configured to generate a semantic parsing result according to the intention of the patient and the standard term; and

an output unit, configured to output a corresponding answer according to the semantic parsing result.

In an embodiment, the intention recognizer is further configured to:

acquire document subject information of the medical consultation sentence input by the patient;

convert the medical consultation sentence input by the patient from text data into vector data;

acquire a score of the medical consultation sentence for each of preset intentions according to the document subject information and the vector data corresponding to the medical consultation sentence; and

determine the intention of the patient according to the score of the medical consultation sentence for each of the preset intentions.

In an embodiment, the substantive term extractor includes:

a template acquisition unit, configured to acquire a semantic slot template corresponding to the intention of the patient, wherein the semantic slot template includes a plurality of semantic slots for representing the disease characteristics; and

a recognition unit, configured to extract substantive terms corresponding to the plurality of semantic slots in the semantic slot template from the medical consultation sentence.

In an embodiment, the recognition unit is further configured to:

perform sequence labeling on the medical consultation sentence by using a sequence labeling model, and acquire the substantive terms corresponding to the plurality of semantic slots in the semantic slot template according to a sequence labeling result.

In an embodiment, the parser includes:

a filling unit, configured to fill the standard term corresponding to the medical consultation sentence of the patient into corresponding one of the plurality of semantic slots;

a determining unit, configured to determine whether there is an unfilled semantic slot in the semantic slot template;

an inquiring unit, configured to, in a case where there is an unfilled semantic slot in the semantic slot template, generate an inquiry question corresponding to the unfilled semantic slot, and fill the unfilled semantic slot according to an answer sentence input by the patient for the inquiry question, until all of the semantic slots in a current semantic slot template are filled; and

a parsing unit, configured to generate the semantic parsing result according to the intention of the patient, each semantic slot, and a filled value of the semantic slot.

In an embodiment, the output unit includes:

a matching degree calculator, configured to calculate a matching degree between the semantic parsing result and each sample group in a doctor-patient question-answering knowledge library, wherein each sample group includes a question sample and its corresponding answer sample; and

an output unit, configured to output an answer sample having a maximum matching degree.

In an embodiment, the matching degree calculator includes:

a calculation subunit, configured to calculate a similarity between the semantic parsing result and the question sample and a correlation between the semantic parsing result and the answer sample; and

a generation subunit, configured to generate the matching degree according to the similarity, a first weighting coefficient, the correlation, and a second weighting coefficient.

In an embodiment, the medical question-answering system further includes:

a standard term library generator, configured to generate a standard term library in which a plurality of standard term samples are stored;

a synonym collector, configured to collect at least one synonym corresponding to each of the plurality of standard term samples;

a filter, configured to calculate a similarity between each standard term sample and its corresponding synonym, maintain each synonym having a similarity greater than a preset value, and remove each synonym having a similarity less than or equal to the preset value; and

a mapping table generator, configured to generate the synonym mapping table according to each synonym and its corresponding synonym which is maintained currently.

A third aspect of the present disclosure provides an electronic device, which includes a processor and a memory which has a computer program stored thereon, wherein the computer program, when executed by the processor, implements the medical question-answering method according to any one of the embodiments of the first aspect of the present disclosure.

A fourth aspect of the present disclosure provides a non-transitory computer readable storage medium on which a computer program is stored, wherein the computer program, when executed by a processor, implements the medical question-answering method according to any one of the embodiments of the first aspect of the present disclosure.

### BRIEF DESCRIPTION OF THE DRAWINGS

The accompanying drawings, which are included to provide a further understanding of the present disclosure and constitute a part of this specification, serve to explain the present disclosure together with the following exemplary embodiments, but are not intended to limit the present disclosure. In the drawings:

FIG. 1 is a schematic flowchart of a medical question-answering method according to an embodiment of the present disclosure;

FIG. 2 is a schematic flowchart of another medical question-answering method according to an embodiment of the present disclosure;

FIG. 3 is a schematic flowchart of generating a synonym mapping table according to an embodiment of the present disclosure;

FIG. 4 is a schematic structural diagram of a medical question-answering system according to an embodiment of the present disclosure; and

FIG. 5 is a schematic structural diagram of a medical question-answering system according to an embodiment of the present disclosure.

### DETAILED DESCRIPTION

To enable one of ordinary skill in the art to better understand technical solutions of the present disclosure, a medical question-answering method, a medical question-answering system, an electronic device, and a computer readable storage medium provided by the present disclosure will be described in detail below with reference to the accompanying drawings.

Exemplary embodiments will be described more fully hereinafter with reference to the accompanying drawings, but may be embodied in different forms and should not be construed as being limited to the forms set forth herein. Rather, these embodiments are provided such that the present disclosure will be thorough and complete, and will fully convey the scope of the present disclosure to one of ordinary skill in the art. As used herein, the term “and/or” includes any and all combinations of one or more of the associated listed items.

The terms used herein are for the purpose of describing particular embodiments only and are not intended to limit the present disclosure. As used herein, the singular forms “a”, “an” and “the” are intended to include the plural forms as well, unless the context clearly indicates otherwise. It should be further understood that the terms “include” and/or “comprise”, when used in this specification, specify the presence of stated features, integers, steps, operations, elements, and/or components, but do not preclude the presence or addition of one or more other features, integers, steps, operations, elements, components, and/or groups thereof.

Embodiments described herein may be described with reference to plan and/or cross-sectional views in idealized representations of the present disclosure. Accordingly, the example illustrations may be modified in accordance with manufacturing techniques and/or tolerances. Accordingly, embodiments are not limited to the embodiments shown in the drawings, but include modifications of configurations formed based on a manufacturing process. Thus, the regions illustrated in the figures have schematic properties, and the shapes of the regions shown in the figures illustrate specific shapes of regions of elements, but are not intended to be limitative.

Unless otherwise defined, all terms (including technical and scientific terms) used herein have the same meaning as commonly understood by one of ordinary skill in the art. It should be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

The inventors of the present disclosure have found that, when a patient (or a user) consults (e.g., asks a question to) a related online disease question-answering website, the related online disease question-answering website (or a related question-answering system) cannot respond well to the question input by the patient because the question input by the patient generally have the problem of colloquialization (e.g., a non-standard term) and diversity of description.

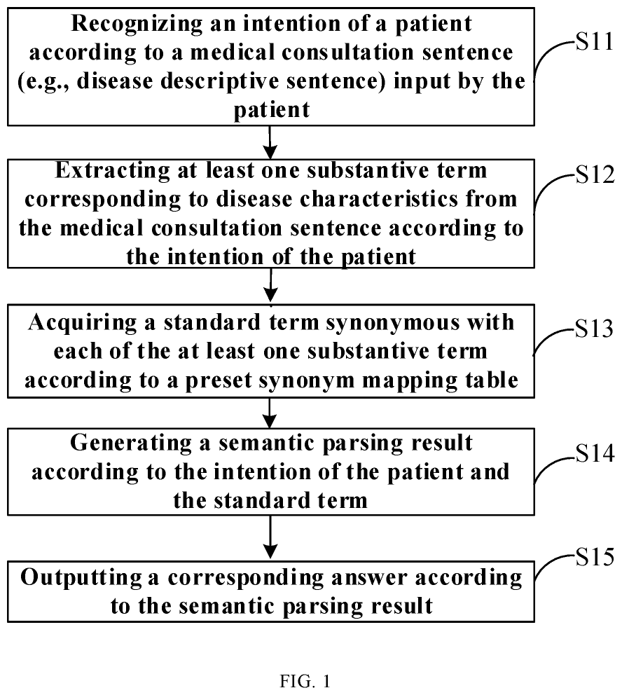

FIG. 1 is a schematic flowchart of a medical question-answering method according to an embodiment of the present disclosure. For example, the medical question-answering method may be performed by a medical question-answering system, which may be implemented by software and/or hardware, and may be integrated in an electronic device. As shown in FIG. 1, the medical question-answering method may include the following steps S11 to S15.

Step S11 may include recognizing an intention of a patient (or a user) according to a medical consultation sentence input by the patient (or the user).

For example, types of the intention of the patient may include “disease diagnosis”, “treatment”, “medication” (e.g., using a drug), “medication effect consultation”, “disease cause consultation”, “surgery consultation”, and the like.

For example, in step S11, a preset intention recognition model may be employed to determine a type of the intention of the patient.

Step S12 may include extracting at least one substantive term corresponding to disease characteristics from the medical consultation sentence according to the intention of the patient.

In some embodiments, the disease characteristics of the patient may include: at least one of an onset symptom, a symptom onset time, a symptom duration, an accompanying symptom, a medical history, a treatment history, and an age of the patient. Each intention may correspond to one or more preset disease characteristics. For example, each substantive term may be a word corresponding to at least one of the onset symptom, the symptom onset time, the symptom duration, the accompanying symptom, the medical history, the treatment history, and the age of the patient.

For example, in a case where the medical consultation sentence input by the patient is “what should an adult do if the adult has had a fever of 38.5 degrees for two days?”, an intention of the patient is recognized as “treatment”. Then, a substantive term “adult” corresponding to “the age of the patient”, a substantive term “fever” corresponding to the “onset symptom”, a substantive term “two days” corresponding to the “symptom duration” and the like are extracted from the medical consultation sentence according to the intention of the patient “treatment”.

Step S13 may include acquiring a standard term synonymous with each of the at least one substantive term according to a preset synonym mapping table. For example, the synonym mapping table may include mapping relationships between a plurality of standard terms and corresponding synonym(s) (i.e., the at least one substantive term).

For example, the at least one substantive term extracted in step S12 may be colloquial terms such as “having loose bowels”, “can't eat”, “poor appetite”, “losing one's appetite” and the like. According to the synonym mapping table, a standard term corresponding to the substantive term “having loose bowels” may be determined to be “diarrhea”, and a standard term corresponding to the substantive terms “can't eat”, “poor appetite” and “losing one's appetite” may be determined to be “anorexia”.

Step S14 may include generating a semantic parsing result according to the intention of the patient and the standard term.

Step S15 may include outputting a corresponding answer according to the semantic parsing result.

The related medical question-answering system cannot accurately determine the true meaning of a question input by a patient and thus cannot answer the question accurately, because the question input by the patient has the problems such as colloquialization and description diversity when consulting. In an embodiment of the present disclosure, some terms (i.e., the at least one substantive term) related to a disease, a symptom and characteristics describing the symptom in the medical consultation sentence input by the patient are extracted, and then are converted into standard terms, thereby facilitating the system to give an accurate answer.

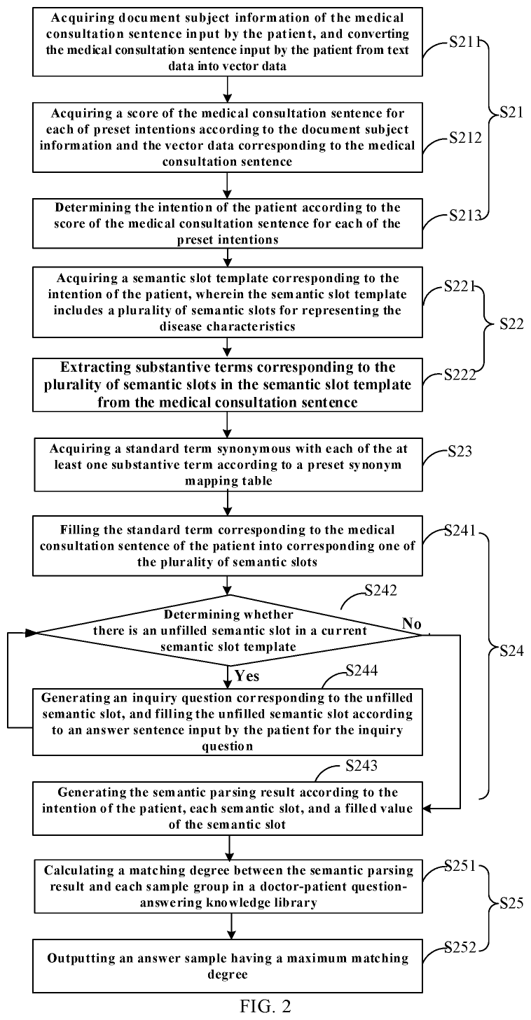

FIG. 2 is a schematic flowchart of another medical question-answering method according to an embodiment of the present disclosure. As shown in FIG. 2, the medical question-answering method may include the following steps S21 to S25.

Step S21 may include recognizing an intention of a patient according to a medical consultation sentence input by the patient.

In some embodiments, step S21 may include the following steps S211 to S213.

Step S211 may include acquiring document subject information of the medical consultation sentence input by the patient, and converting the medical consultation sentence input by the patient from text data into vector data.

Optionally, the document subject information of the medical consultation sentence may be generated by using a document subject generation (which is also referred to as Latent Dirichlet Allocation that is abbreviated as LDA) model, and the medical consultation sentence may be converted into an embedding word (or word embedding) vector by using a word2vec model.

Step S212 may include acquiring a score of the medical consultation sentence for each of preset intentions according to the document subject information and the vector data corresponding to the medical consultation sentence.

For example, the document subject information and the vector data corresponding to the medical consultation sentence may be concatenated to acquire (or obtain) a vector matrix including word information and subject information, and the vector matrix may be input to a bidirectional gated recurrent unit (BiGRU) to acquire the score of the medical consultation sentence for each of the preset intentions. Each of the preset intentions may be acquired according to a manner of pre-learning.

Step S213 may include determining the intention of the patient according to the score of the medical consultation sentence for each of the preset intentions.

For example, the score for each of the preset intentions is mapped to a probability in the range of 0 to 1 (i.e., a probability greater than 0 and less than 1) by using a softmax classifier (i.e., a classifier referred to as softmax), thereby determining the intention of the patient according to the maximum probability. Here, the softmax classifier is merely an example, and other classifiers such as a svm classifier (i.e., a classifier referred to as svm) may be applicable.

For example, step S21 may be performed by using a preset intention recognition model, which may include the word2vec model, the document subject generation (LDA) model, the bidirectional gated recurrent unit (BiGRU), and the softmax classifier.

The intention recognition model for a desired function may be acquired by a training method. In training, medical consultation sentences of patients may be extracted from samples, for example, from doctor-patient questioning and answering data collected from a professional medical website or App (such as the website of online HaoDaiFu at www.haodf.com, the website of DingXiangYiSheng at www.dxy.com, the website of PingAnHaoDaiFu at www.jk.cn, and/or the like) or a medical inquiry history (i.e., the records of inquiry between patients and doctors), and texts of the medical consultation sentences may be subjected to data cleaning (i.e., a non-key word such as “hello” or the like in the texts is removed). Next, text data may be clustered by using a clustering algorithm, and a type of an intention of a general question of a patient may be determined by using a sampling method. Then, the type of each intention may be determined by an expert (e.g., a doctor or a professional staff with medical knowledge), and the intention recognition model may be trained according to each medical consultation sentence and the type of intention corresponding to the medical consultation sentence.

Taking Table 1 as an example, an example in which some collected medical consultation sentences and their corresponding intention types (or intentions) is shown.

TABLE 1

| | Type of |
|---|---|
| Medical consultation sentence | intention |
| What should I do about the condition | Treatment |
| that the inflammation of severe | |
| pancreatitis does not improve? | |
| I want to determine whether there is | Medication effect |
| ovulation, after taking Letrozole for | consultation |
| polycystic ovary syndrome. | |
| Red rashes appeared successively on | Disease diagnosis |
| various parts of the body. | |
| A time period and medication for | Consultation on |
| curing lumbar muscle strain. | medication |
| How did this happen and what caused | Disease cause |
| the fever? | consultation |
| Are the symptoms of squeezing eyes, | Disease diagnosis |
| opening mouth, and nodding serious? | |
| There is no menstruation, after taking | Medication effect |
| progesterone for polycystic ovary | consultation |
| syndrome for seven days. | |
| What should I do if a child is needed in | Treatment |
| a case of a multi-ovary ovarian cyst? | |
| What drug should be used for treating | Consultation on |
| frictional lichen on a | medication |
| two-and-a-half-year-old baby? | |
| Can I make dental implant if multiple | Surgery |
| teeth have been missing? | consultation |

In some embodiments, the types of intent may include: “disease diagnosis”, “treatment”, “consultation on medication” (e.g., consultation on using a drug), “medication effect consultation”, “disease cause consultation”, “surgery consultation”, and “other”. In a case where an intention of a patient is determined to be “other” according to the intention recognition model, the patient be directly prompted that the system cannot answer the input question.

Step S22 may include extracting at least one substantive term corresponding to disease characteristics from the medical consultation sentence according to the intention of the patient.

For example, the disease characteristics include: at least one of an onset symptom, a symptom onset time, a symptom duration, an accompanying symptom, a medical history, a treatment history, and an age of the patient.

In some embodiments, step S22 may include the following steps S221 and S222.

Step S221 may include acquiring semantic slot templates corresponding to the intention of the patient, each of the semantic slot templates including a plurality of semantic slots for representing the disease characteristics.

For example, the semantic slot templates corresponding to each intention may be preset. For example, the semantic slot templates corresponding to the intention of “medication consultation” may include a plurality of semantic slots for representing “symptom”, “symptom onset time”, “accompanying symptom”, “medical history”, and “treatment history”.

Step S222 may include extracting substantive terms corresponding to the semantic slots in the semantic slot template from the medical consultation sentence.

In some embodiments, a named entity recognition method may be employed to extract the substantive terms corresponding to the semantic slots in the semantic slot template from the medical consultation sentence. Specifically, step S222 may include: performing sequence labeling on the medical consultation sentence by using a sequence labeling model, and acquiring the substantive terms corresponding to the semantic slots in the semantic slot template according to a sequence labeling result.

For example, the sequence labeling model may be a BilSTM-CRF model that employs a BIO annotation set to perform the named entity recognition based on names of semantic slots. For example, the semantic slot template may include two semantic slots of “disease” and “symptom name”, and when labeling is performed by adopting the BIO annotation set, B-DIS represents a first word of the disease, I-DIS represents a non-first word of the disease, B-SYM represents a first word of the symptom, I-SYM represents a non-first word of the symptom, and O represents that a word does not belong to a portion of the named entity. Alternatively, in a case where the semantic slot template includes another number (i.e., quantity) of semantic slots such as: “disease”, “disease onset time”, “medication history” and “symptom name”, B1-DIS may represent a first word of the disease, I1-DIS may represent a non-first word of the disease, B1-SYM may represent a first word of the symptom, I1-SYM may represent a non-first word of the symptom, B2-DIS may a first word of the disease onset time, I2-DIS may represent a non-first word of the disease onset time, B1-SYM may represent a first word of the medication history, I2-SYM may represent a non-first word of the symptom; and O may represent that a word does not belong to a portion of the named entity.

For example, the BilSTM-CRF model may be acquired by training. During training, a plurality of sample sequences and labeling sequences respectively corresponding to the plurality of sample sequences may be provided, and each of the sample sequences has the same length as a length of the labeling sequence corresponding to this sample sequence. Then, the sample sequences are taken as an input of an initial BilSTM-CRF model, the labeling sequences corresponding to the sample sequences are taken as an output of the initial BilSTM-CRF model, and the BilSTM-CRF model with the required functions is acquired through multiple times of training.

Step S23 may include acquiring a standard term synonymous with each of the at least one substantive term according to the preset synonym mapping table. For example, the synonym mapping table may include mapping relationships between a plurality of standard terms and corresponding synonym(s) (i.e., the at least one substantive term).

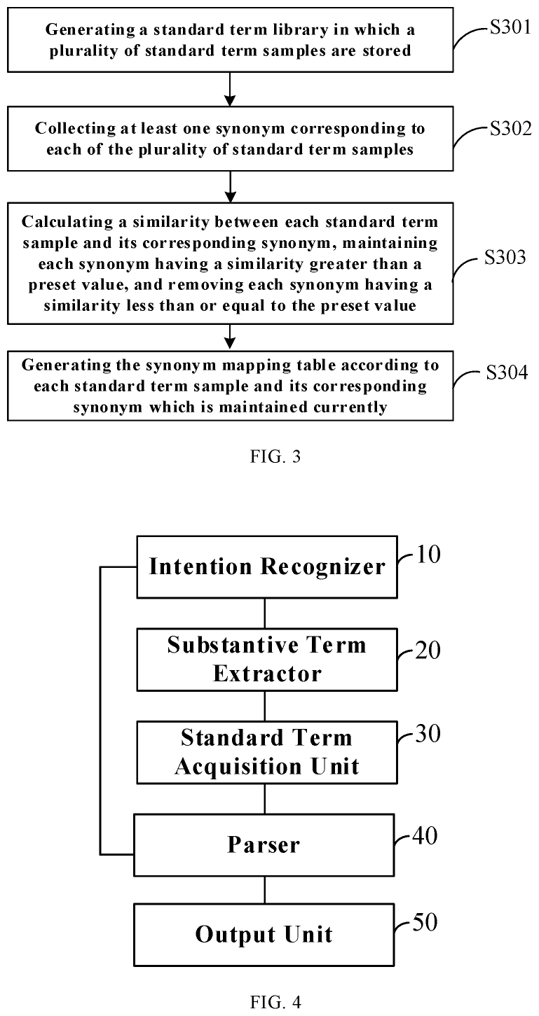

For example, the synonym mapping table may be provided prior to performing step S21. FIG. 3 is a schematic flowchart of generating the synonym mapping table according to an embodiment of the present disclosure. As shown in FIG. 3, the process of generating the synonym mapping table may include the following steps S301 to S304.

Step S301 may include generating a standard term library in which a plurality of standard term samples (i.e., samples of a plurality of standard terms) are stored.

S302 may include collecting at least one synonym corresponding to each of the standard term samples.

For example, the synonym corresponding to each of the standard term samples may refer to a term having the same or substantially the same meaning as that of the standard term. The at least one synonym corresponding to each of the standard term samples may be collected from websites such as large medical websites, forums, Baidu Encyclopedia (see website baike.baidu.com), and the collected synonym(s) in this step may be colloquial and non-standard term(s).

For example, the standard terms may be acquired from an authoritative medical textbook, dictionary, manual, etc., such as medical guidelines for various diseases issued by medical health management departments, clinical medical guidelines issued by medical association, a Physician's Desk Reference (PDR), pharmacopeia, etc.

Step S303 may include calculating a similarity (such as a cosine similarity) between each standard term sample and its corresponding synonym, maintaining (or retaining) each synonym having a similarity greater than a preset value, and removing each synonym having a similarity less than or equal to the preset value.

For example, a similarity may be calculated by using an existing synonym recognition model (e.g., word2vec may be used for calculating a semantic similarity between terms). When the similarity is too small, it is indicated that a standard term sample has a meaning different from that of the corresponding collected synonym, and the collected synonym may be removed (or canceled). For example, the similarity may be a value between 0 and 1.

For example, the preset value for determining the similarity between each standard term sample and its corresponding synonym may be set according to the requirement of a practical application.

In the field of natural language processing technology, many models for recognizing synonyms have been developed, such as a toolkit referred to as Synonyms, an LRWE model, etc. An embodiment of the present disclosure may also adopt these known models to recognize synonyms.

Step S304 may include generating the synonym mapping table according to each standard term sample (i.e., the sample of each standard term) and its corresponding synonym which is maintained currently.

Table 2 schematically shows a portion of a synonyms mapping table.

TABLE 2

| Standard term sample | Synonym | Similarity |
|---|---|---|
| fever | pyrexia | 0.9742406010627747 |
| abdominal pain | stomach ache | 0.9563860893249512 |
| headache | dizziness | 0.9934560656547546 |
| arthralgia of | a little pain in one | 0.8579318046569824 |
| finger | finger joint | |
| arthralgia of | pain in a joint of a left | 0.8957708954811096 |
| finger | thumb | |
| arthralgia of | pain in different joints | 0.8284000039100647 |
| finger | of two fingers | |
| arthralgia of | there is a little pain in a | 0.7430312812328338 |
| finger | finger joint for no | |
| | reason | |

In step S23, a standard term synonymous with a substantive term may be directly looked up from the synonym mapping table.

Step S24 may include generating a semantic parsing result according to the intention of the patient and the standard term.

In some embodiments, step S24 may include the following steps S241 to S244.

Step S241 may include filling the standard terms corresponding to the medical consultation sentence of the patient into corresponding semantic slots of the plurality of semantic slots of the current semantic slot template.

For example, the medical consultation sentence input by the patient is “what kind of drug should be taken for having a cold and a dry throat”, and the intention of the patient may be recognized as “medication consultation” according to the medical consultation sentence. The plurality of semantic slots in the semantic slot template corresponding to the intention include: “symptom”, “symptom onset time”, “accompanying symptom”, “medical history”, and “treatment history”. By performing the named entity recognition on the medical consultation sentence, the substantive term corresponding to the “symptom” is acquired as “having a cold and a dry throat”. A standard term corresponding to “dry throat” is acquired as “dryness of the throat” by using the synonym mapping table, and the standard term “having a cold and a dryness of the throat” is filled into the semantic slot for the “symptom”.

Step S242 may include determining whether there is an unfilled semantic slot in the current semantic slot template.

If a result of the determination indicates that there is no unfilled semantic slot in the current semantic slot template, the method proceeds to step S243, as described below.

If the result of the determination indicates that there is an unfilled semantic slot in the current semantic slot template, the method proceeds to step S244. In step S244, an inquiry question corresponding to the unfilled semantic slot is generated, and the unfilled semantic slot is filled according to an answer sentence input by the patient for the inquiry question until all of the semantic slots are filled.

In some practical application scenarios, a medical consultation sentence input by a patient for the first time may contain only a few disease characteristics, for example, only a symptom and a disease onset time. However, in most cases, a likelihood that a patient has a particular disease is directly determined according to the time, nature, state, and accompanying symptoms of symptom onset of the patient. For example, emesis is a common symptom which may be resulted from a cold or another reason, and a diagnosed disease may vary depending on different vomiting times. In an embodiment of the present disclosure, when useful information in the medical consultation sentence of the user is not enough to completely fill all of the semantic slots in the semantic slot template, the inquiry question may be output to the user (or the patient), to acquire more comprehensive information. In this way, the medical question-answering method according to an embodiment of the present disclosure goes beyond the traditional single-turn question-answering mode and achieves multi-turn interaction.

Step S243 may include generating the semantic parsing result according to the intention of the patient, each semantic slot, and a filled value of the semantic slot.

For example, the semantic parsing result may be in the form of triplets of act (slot1=value1, slot2=value2 . . . ), where act represents an intention, slot1 and slot2 represent semantic slots, and value1 and value2 represent slot values filled in the semantic slots. For example, the intention may be “medication consultation”, and the semantic slots may include “symptom”, “symptom onset time”, “accompanying symptom”, “medical history”, and “treatment history”. A slot value of the semantic slot “symptom” may be “headache”, a slot value of the semantic slot “symptom onset time” may be “one day ago”, a slot value of the semantic slot “accompanying symptom” may be “retching”, a slot value of the semantic slot “medical history” may be “HBsAg, HBeAg and HBcAb test positive”, and a slot value of the semantic slot “treatment history” may be “antivirus”. Thus, the semantic parsing result in the form of triplets is as follows: “medication consultation (symptom=headache, symptom onset time=one day ago, accompanying symptom=retching, medical history=HBsAg, HBeAg and HBcAb test positive, treatment history=antivirus)”.

Step S25 may include outputting a corresponding answer according to the semantic parsing result.

In some embodiments, step S25 may include the following steps S251 and S252.

Step S251 may include calculating a matching degree between the semantic parsing result and each sample group in a doctor-patient question-answering knowledge library, each sample group including a question sample (i.e., a sample of a question) and its corresponding answer sample (i.e., a sample of an answer).

For example, step S251 may include the following steps S251a and S251b.

Step S251a may include calculating a similarity between the semantic parsing result and the question sample, and a correlation between the semantic parsing result and the answer sample. For example, the similarity between the semantic parsing result and the question sample and the correlation between the semantic parsing result and the answer sample may be calculated by using an existing correlation calculation method, such as a BM25 algorithm.

Step S251b may include generating the matching degree according to the similarity and a first weighting coefficient, and the correlation and a second weighting coefficient. That is, the matching degree is: a sum of a product of the similarity and the first weighting coefficient and a product of the correlation and the second weighting coefficient.

For example, the first weighting coefficient and the second weighting coefficient may be set according to a practical requirement, each of the first weighting coefficient and the second weighting coefficient is between 0 and 1, and a sum of the first weighting coefficient and the second weighting coefficient is equal to 1.

Step S252 may include outputting an answer sample having the maximum matching degree.

Since the matching degree is a weighted sum of the similarity between the semantic parsing result and the question sample and the correlation between the semantic parsing result and the answer sample, in the sample group with the maximum matching degree, the similarity between the question sample and the semantic parsing result and the correlation between the answer sample and the semantic parsing result are both high.

Alternatively, other methods may be used for selecting an answer corresponding to the semantic parsing result. For example, if the similarity between the semantic parsing result and a question sample exceeds a preset first threshold, and if the correlation between the semantic parsing result and an answer sample corresponding to the question sample exceeds a second threshold, the answer sample may be output.

Table 3 shows a similarity between a question corresponding to a semantic parsing result and each question sample and a correlation between the question corresponding to the semantic parsing result and each answer sample.

For example, the question sample and the answer sample in a same row in Table 3 belong to a same sample group. For the medical consultation sentence “Can I make dental implant if multiple teeth have been missing?” of a patient in Table 3, the similarity between the semantic parsing result thereof and the first question sample and the correlation between the semantic parsing result and the first answer sample both are the maximum values, and in this case, the matching degree between the semantic parsing result and the first sample group is the highest. Thus, the first answer sample will be output.

TABLE 3

| Ques- | Problem | Simi- | | Cor- |
|---|---|---|---|---|
| tion | sample | larity | Answer sample | relation |
| Can I | Can I | 0.951 | Hello, dental implant may be | 0.900 |
| make | make | | made for multiple teeth having | |
| dental | dental | | been missing. Timely treatment | |
| implant | implant in | | after missing teeth may not only | |
| if | case of | | restore the chewing function but | |
| multiple | multiple | | also prevent adjacent teeth from | |
| teeth | teeth | | being affected, and further | |
| have | having | | prevent maxillofacial aesthetics | |
| been | been | | from being affected. Dental | |
| miss- | missing? | | implants have been referred to as | |
| ing? | | | “the third set of human teeth”, | |
| | | | and the effect of dental implant is | |
| | | | excellent. | |
| | Can I | 0.855 | Relevant checks are required to | 0.791 |
| | make | | determine whether dental implant | |
| | dental | | is possible. | |
| | implant if | | | |
| | a tooth has | | | |
| | been | | | |
| | missing? | | | |
| | What is a | 0.682 | The loss of a tooth firstly | 0.600 |
| | result of | | affects the beauty of personal | |
| | missing a | | appearance. The loss of a | |
| | tooth and | | plurality of teeth may cause the | |
| | no dental | | face to sink, the chewing function | |
| | implant? | | to decline, and alveolar bones to | |
| | | | slowly shrink easily. Since the | |
| | | | loss of the teeth at two sides will | |
| | | | cause the original supporting | |
| | | | force to be lost, adjacent teeth | |
| | | | may incline, then the adjacent | |
| | | | teeth will be loosened and finally | |
| | | | fall off. Thus, sufficient attention | |
| | | | should be paid thereto. | |
| | Can dental | 0.532 | Although dental implant has | 0.480 |
| | implant | | many advantages, it cannot be | |
| | solve the | | performed once and work for | |
| | problem | | ever. If a doctor's instructions on | |
| | of missing | | oral hygiene are not followed | |
| | a tooth? | | after dental implant, the lifetime | |
| | | | of the dental implants will be | |
| | | | shortened, making the dental | |
| | | | implants that can work for 20 | |
| | | | years can only work for 10 years | |
| | | | or less. Of course, if carefully | |
| | | | maintained, the dental implants | |
| | | | work for the rest of your life. | |

The medical question-answering method will be further described below by way of an example thereof.

For example, the medical consultation sentences input by the patient may be “I have caught a cold with blocked nose, headache, dry throat, ache back, tingled temple. I started to get sick yesterday morning, probably with runny nose at first, next a little headache yesterday afternoon, then dry throat and slightly tumbling stomach yesterday evening, and finally insomnia last night until two o'clock or later today morning. I woke up this morning with clear snot in one nostril and a little yellow snot in the other nostril, and after the nose was wiped three or four times, the yellow snot turned into clear snot. I was sweating while having a fever this afternoon. What a drug should I take?”. Firstly, an intention of the patient is recognized as “medication consultation” by using the intention recognition model, and the corresponding semantic slot template includes the following semantic slots of symptom, symptom onset time, accompanying symptom, medical history, and treatment history. Substantive terms corresponding to the disease characteristics in the medical consultation sentences are extracted, and are converted into standard terms. The standard terms are filled into corresponding ones of the plurality of semantic slots in the current semantic slot template to obtain the following: symptom=“I have caught a cold with blocked nose, headache, dry throat, ache back, tingled temple”, symptom onset time=“yesterday morning”, accompanying symptom=“running nose, sweating while having a fever”. Next, an inquiry sentence “presence or absence of medical history” corresponding to the “medical history” is generated, and an inquiry sentence “presence or absence of treatment history” corresponding to the “treatment history” is generated. An answer of the user may be: “having BsAg, HBeAg and HBcAb tested positive, having been on antiviral treatment”, and in this case, the slot value “BsAg, HBeAg and HBcAb tested positive” is filled in the semantic slot of “medical history”, the slot value “antiviral treatment” is filled in the semantic slot of “treatment history”, thereby acquiring a semantic parsing result. Finally, according to the semantic parsing result, an answer is made that “You are suffered from a cold (which is a self-healing disease), and the medication suggestion is as follows: Tylenol, isatis root”.

FIG. 4 is a schematic diagram showing a structure of a medical question-answering system according to an embodiment of the present disclosure, and the medical question-answering system may be used for implementing the medical question-answering method as described above. As shown in FIG. 4, the medical question-answering system may include: an intention recognizer 10, a substantive term extractor 20, a standard term acquisition unit 30, a parser 40, and an output unit 50.

For example, the intention identifier 10 may recognize the intention of the patient according to the medical consultation sentence input by the patient.

In some embodiments, the intention recognizer 10 may convert the medical consultation sentence input by the patient from text data to vector data, and input the vector data to a preset intention recognition model, to recognize the intention of the patient.

In some embodiments, the intent recognition model is a classification model based on a document subject generation model and the bidirectional gated recurrent unit.

In some embodiments, the intention recognizer 10 may further: acquire the document subject information of the medical consultation sentence input by the patient, convert the medical consultation sentence input by the patient from text data into vector data, acquire a score of the medical consultation sentence for each of preset intentions according to the document subject information and the vector data corresponding to the medical consultation sentence, and determine an intention of the patient according to the score of the medical consultation sentence for each of the preset intentions.

The substantive term extractor 20 may extract at least one substantive term corresponding to disease characteristics from the medical consultation sentence according to the intention of the patient. Optionally, the disease characteristics may include: at least one of an onset symptom, a symptom onset time, a symptom duration, an accompanying symptom, a medical history, a treatment history, and an age of the patient.

The standard term acquisition unit 30 may acquire a standard term synonymous with each of the at least one substantive term according to the preset synonym mapping table. For example, the synonym mapping table may include mapping relationships between a plurality of standard terms and corresponding synonym(s) (i.e., the at least one substantive term). The parser 40 may generate a semantic parsing result according to the intention of the patient and the standard term.

The output unit 50 may output a corresponding answer according to the semantic parsing result.

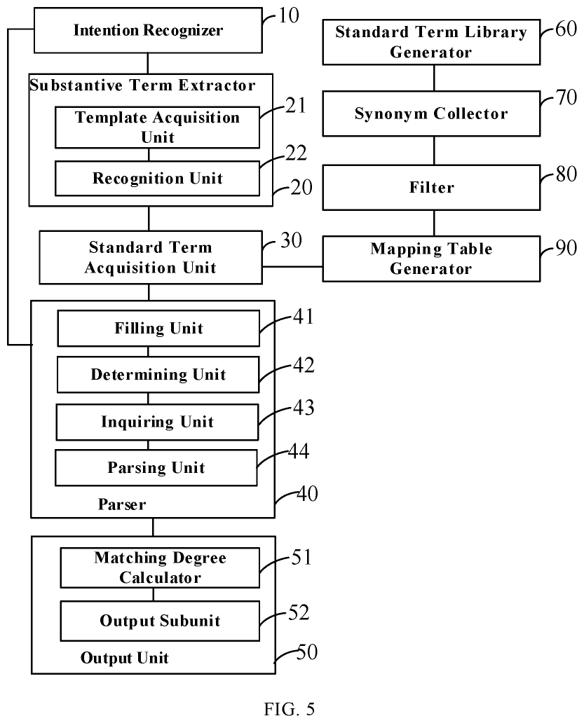

FIG. 5 is a schematic diagram showing a structure of a medical question-answering system according to an embodiment of the present disclosure. As shown in FIG. 5, the medical question-answering system includes a standard term library generator 60, a synonym collector 70, a filter 80, and a mapping table generator 90, in addition to the intention recognizer 10, the substantive term extractor 20, the standard term acquisition unit 30, the parser 40, and the output unit 50.

In some embodiments, the substantive term extractor 20 includes a template acquisition unit 21 and a recognition unit 22.

The template acquisition unit 21 may acquire semantic slot templates corresponding to the intention of the patient, each of the semantic slot templates including a plurality of semantic slots for representing the disease characteristics.

The recognition unit 22 may extract substantive terms corresponding to the semantic slots in the semantic slot template from the medical consultation sentence.

For example, the recognition unit 22 may perform sequence labeling on the medical consultation sentence by using a sequence labeling model, and acquire the substantive terms corresponding to the semantic slots in the semantic slot template according to a sequence labeling result.

Further, the recognition unit may further perform sequence labeling on the medical consultation sentences by using the sequence labeling model, and acquire substantive terms corresponding to the plurality of semantic slots in the semantic slot template according to the sequence labeling result.

In some embodiments, the parser 40 includes: a filling unit 41, a determining unit 42, an inquiring unit 43 and a parsing unit 44.

For example, the filling unit 41 may fill the standard terms corresponding to the medical consultation sentence of the patient into corresponding semantic slots of the plurality of semantic slots of the current semantic slot template.

The determining unit 42 may determine whether there is an unfilled semantic slot in the current semantic slot template.

The inquiring unit 43 may, in a case where there is an unfilled semantic slot in the current semantic slot template, generate an inquiry question corresponding to the unfilled semantic slot, and fill the unfilled semantic slot according to an answer sentence input by the patient for the inquiry question until all of the semantic slots in the current semantic slot template are filled.

The parsing unit 44 may generate the semantic parsing result according to the intention of the patient, each semantic slot, and a filled value of the semantic slot.

In some embodiments, the output unit 50 includes a matching degree calculator 51 and an output subunit 52.

For example, the matching degree calculator 51 may calculate a matching degree between the semantic parsing result and each sample group in a doctor-patient question-answering knowledge library, each sample group including a question sample and its corresponding answer sample.

In some embodiments, the matching degree calculator 51 includes: a calculation subunit 511 and a generation subunit 512.

For example, the calculation subunit 511 may calculate a similarity between the semantic parsing result and the question sample, and a correlation between the semantic parsing result and the answer sample.

The generation subunit 512 may generate the matching degree according to the similarity and a first weighting coefficient as well as the correlation and a second weighting coefficient.

The output unit 52 may output an answer sample having the maximum matching degree.

The standard term library generator 60 may generate a standard term library, in which a plurality of standard term samples are stored.

The synonym collector 70 may collect at least one synonym corresponding to each of the standard term samples.

The filter 80 may calculate a similarity between each standard term sample and its corresponding synonym, maintain each synonym having a similarity greater than the preset value, and remove each synonym having a similarity less than or equal to the preset value. For example, the preset value may be 0.5, 0.6, 0.7, 0.8, 0.9, or the like.

The mapping table generator 90 may generate the synonym mapping table according to each standard term sample and its corresponding synonym which is maintained currently.

For the description of the implementation details and the technical effects of the above components and units, reference may be made to the description of the foregoing method embodiments, which are not repeated here.

It should be understood that, the medical question-answering system shown in FIG. 4 or 5 may be a single computer or a single computing device, or may be a plurality of computers or a plurality of computing devices, which are connected to each other through a wired network and/or a wireless network. In addition, the components of the medical question-answering system shown in FIG. 4 or FIG. 5 may be implemented by hardware, or by a combination of hardware and software. For example, the components of the medical question-answering system shown in FIG. 4 or 5 may be implemented by a central processing unit (CPU), an application processor (AP), a digital signal processor (DSP), a field programmable gate array (FPGA), a microprocessor (MCU), an integrated circuit (IC), or an application specific integrated circuit (ASIC), which has respective functions described in the foregoing embodiments of the present disclosure. For example, the components of the medical question-answering system shown in FIG. 4 or 5 may be implemented by a combination of a processor, a memory, and a computer program stored in the memory, and the processor may read and execute the computer program from the memory to function (or serve) as the respective components of the medical question-answering system shown in FIG. 4 or 5.

The embodiments of the present disclosure also provide an electronic device, which includes one or more processors and a storage device, the storage device has one or more programs stored therein, and when executed by the one or more processors, the one or more programs cause the one or more processors to implement the medical question-answering method according to any one of the foregoing embodiments.

The embodiments of the present disclosure also provide a non-transitory computer readable storage medium having a computer program stored thereon, and when executed by a processor, the computer program implements the medical question-answering method according to any one of the foregoing embodiments.

It should be understood that, the above-mentioned word Embedding technique, word2vec model, softmax classifier, LDA model, BiLSTM (Bi-directional Long Short-Term Memory) model, bidirectional gated recurrent unit (BiGRU), BiLSTM-CRF model, BIO annotation set, BM25 algorithm, etc. are all known techniques in the fields of artificial intelligence and natural language processing. For example, further information on the word Embedding technique and word2vec model may be found in Mikolov T, Chen K, Corrado G S, et al., Efficient Estimation of Word Representations in Vector Space[C], international conference on learning representations, 2013, and the codes of Google open source at the website https://code.google.com/p/word2vec/. Further information on the softmax classifier may be found at the website https://pytorch.org/docs/master/generated/torch.nn.Softmax.html. For further information on the LDA model, reference may be made to Blei, D. M., Ng, A. Y., & Jordan, M. I. (2003), Latent dirichlet allocation. Journal of machine Learning research, 3 (January), 993-1022. For further information on the BilSTM (Bi-directional Long Short-Term Memory) model, reference may be made to Hochreiter S, Schmidhuber J. Long short-term memory[J], Neural Computation, 1997, 9(8): 1735-1780. For further information on bidirectional gated recurrent unit (BiGRU), reference may be made to Cho K, Van Merrienboer B, Gulcehre C, et al, Learning Phrase Representations using RNN Encoder-Decoder for Statistical Machine Translation[J], arXiv: Computation and Language, 2014. For further information on the BilSTM-CRF model, reference may be made to Huang Z, Xu W, Yu K, et al, Bidirectional LSTM-CRF Models for Sequence Tagging.[J], arXiv: Computation and Language, 2015. For further information on the BIO annotation set, reference may be made to Sang E F, De Meulder F, Introduction to the CoNLL-2003 shared task: language-independent named entity recognition[C], north American chapter of the association for computational linguistics, 2003: 142-147. Further information on the BM25 algorithm may be found in the book “Introduction to Information Retrieval”.

It will be understood by one of ordinary skill in the art that, all or some of the steps of each method and all or some of the functional components/units in each device or system disclosed above may be implemented as software, firmware, hardware, or suitable combinations thereof. In a hardware implementation, the division of functional components/units mentioned in the foregoing description may not necessarily correspond to the division of physical components. For example, one physical component may have multiple functions, or one function or step may be performed by several physical components in cooperation. Some or all of the physical components may be implemented as software executed by a processor such as a central processing unit (CPU), a digital signal processor (DSP), a field programmable gate array (FPGA) or a microprocessor (MCU), or implemented as hardware, or implemented as an integrated circuit such as an application specific integrated circuit (ASIC). Such software may be distributed on a computer readable storage medium, which may include a computer storage medium (or non-transitory computer readable storage medium) or a communication medium (or a transitory medium). The term of computer storage medium includes a volatile or nonvolatile, removable or non-removable medium implemented by any method or technology for storage of information (such as computer readable instructions, data structures, program modules or other data), as is well known to one of ordinary skill in the art. The computer storage medium includes, but is not limited to, RAM, ROM, EEPROM, flash memory or other memory technology, CD-ROM, digital versatile disk (DVD) or other optical disk storage, magnetic cassette, magnetic tape, magnetic disk storage or other magnetic storage device, or any other medium which can be used for storing the desired information and which can be accessed by a computer. In addition, the communication medium generally includes computer readable instructions, data structures, program modules or other data in a modulated data signal such as a carrier wave or other transport mechanism, and may include any information delivery medium, as is well known to one of ordinary skill in the art.

Exemplary embodiments have been disclosed herein, and specific terms are employed. However, they are employed and should be interpreted in a generic and descriptive sense only and not for purposes of limitation. In some instances, features, characteristics and/or elements described in connection with a particular embodiment may be used alone or in combination with features, characteristics and/or elements described in connection with other embodiments, unless expressly stated otherwise, as would be apparent to one of ordinary skill in the art. It will therefore be understood by one of ordinary skill in the art that various changes in form and details may be made therein without departing from the scope of the present disclosure as set forth in the appended claims.

It will be understood that, the above embodiments are merely exemplary embodiments employed to explain the principles of the present disclosure, and the present disclosure is not limited thereto. It will be apparent to one of ordinary skill in the art that various changes and modifications may be made without departing from the scope of the present disclosure as defined in the appended claims, and such changes and modifications also fall within the scope of the present disclosure.

1. A medical question-answering method, comprising:
recognizing an intention of a patient according to a medical consultation sentence input by the patient;
extracting at least one substantive term corresponding to disease characteristics from the medical consultation sentence according to the intention of the patient;
acquiring a standard term synonymous with each of the at least one substantive term according to a preset synonym mapping table, wherein the synonym mapping table comprises mapping relationships between a plurality of standard terms and corresponding substantive terms;
generating a semantic parsing result according to the intention of the patient and the standard term; and
outputting a corresponding answer according to the semantic parsing result.

2. The medical question-answering method according to claim 1, wherein the recognizing an intention of a patient according to a medical consultation sentence input by the patient comprises:
acquiring document subject information of the medical consultation sentence input by the patient;
converting the medical consultation sentence input by the patient from text data into vector data;
acquiring a score of the medical consultation sentence for each of preset intentions according to the document subject information and the vector data corresponding to the medical consultation sentence; and
determining the intention of the patient according to the score of the medical consultation sentence for each of the preset intentions.

3. The medical question-answering method according to claim 1, wherein the extracting at least one substantive term corresponding to disease characteristics from the medical consultation sentence according to the intention of the patient comprises:
acquiring a semantic slot template corresponding to the intention of the patient, wherein the semantic slot template comprises a plurality of semantic slots for representing the disease characteristics; and
extracting substantive terms corresponding to the plurality of semantic slots in the semantic slot template from the medical consultation sentence.

4. The medical question-answering method according to claim 3, wherein the extracting substantive terms corresponding to the plurality of semantic slots in the semantic slot template from the medical consultation sentence comprises:
performing sequence labeling on the medical consultation sentence by using a sequence labeling model, and acquiring the substantive terms corresponding to the plurality of semantic slots in the semantic slot template according to a sequence labeling result.

5. The medical question-answering method according to claim 3, wherein the generating a semantic parsing result according to the intention of the patient and the standard term comprises:
filling the standard term corresponding to the medical consultation sentence of the patient into corresponding one of the plurality of semantic slots;
determining whether there is an unfilled semantic slot in a current semantic slot template;
in a case where a result of determination indicates that there is an unfilled semantic slot in the current semantic slot template, generating an inquiry question corresponding to the unfilled semantic slot, and filling the unfilled semantic slot according to an answer sentence input by the patient for the inquiry question, until all of the semantic slots in the current semantic slot template are filled; and
generating the semantic parsing result according to the intention of the patient, each semantic slot, and a filled value of the semantic slot.

6. The medical question-answering method according to claim 1, wherein the outputting a corresponding answer according to the semantic parsing result comprises:
calculating a matching degree between the semantic parsing result and each sample group in a doctor-patient question-answering knowledge library, wherein each sample group comprises a question sample and its corresponding answer sample; and
outputting an answer sample having a maximum matching degree.

7. The medical question-answering method according to claim 6, wherein the calculating a matching degree between the semantic parsing result and each sample group in a doctor-patient question-answering knowledge library comprises:
calculating a similarity between the semantic parsing result and the question sample and a correlation between the semantic parsing result and the answer sample; and
generating the matching degree according to the similarity, a first weighting coefficient, the correlation, and a second weighting coefficient.

8. The medical question-answering method according to claim 1, wherein the disease characteristics comprises at least one of an onset symptom, a symptom onset time, a symptom duration, an accompanying symptom, a medical history, a treatment history, and an age of the patient.

9. The medical question-answering method according to claim 1, wherein prior to the recognizing an intention of a patient according to a medical consultation sentence input by the patient, the medical question-answering method further comprises:
generating a standard term library in which a plurality of standard term samples are stored;
collecting at least one synonym corresponding to each of the plurality of standard term samples;
calculating a similarity between each standard term sample and its corresponding synonym, maintaining each synonym having a similarity greater than a preset value, and removing each synonym having a similarity less than or equal to the preset value; and
generating the synonym mapping table according to each standard term sample and its corresponding synonym which is maintained currently.

10. A medical question-answering system, comprising:
an intention recognizer, configured to recognize an intention of a patient according to a medical consultation sentence input by the patient;
a substantive term extractor, configured to extract at least one substantive term corresponding to disease characteristics from the medical consultation sentence according to the intention of the patient;
a standard term acquisition unit, configured to acquire a standard term synonymous with each of the at least one substantive term according to a preset synonym mapping table, wherein the synonym mapping table comprises mapping relationships between a plurality of standard terms and corresponding substantive terms;
an parser, configured to generate a semantic parsing result according to the intention of the patient and the standard term; and
an output unit, configured to output a corresponding answer according to the semantic parsing result.

11. The medical question-answering system according to claim 10, wherein the intention recognizer is further configured to:
acquire document subject information of the medical consultation sentence input by the patient;
convert the medical consultation sentence input by the patient from text data into vector data;
acquire a score of the medical consultation sentence for each of preset intentions according to the document subject information and the vector data corresponding to the medical consultation sentence; and
determine the intention of the patient according to the score of the medical consultation sentence for each of the preset intentions.

12. The medical question-answering system according to claim 10, wherein the substantive term extractor comprises:
a template acquisition unit, configured to acquire a semantic slot template corresponding to the intention of the patient, wherein the semantic slot template comprises a plurality of semantic slots for representing the disease characteristics; and
a recognition unit, configured to extract substantive terms corresponding to the plurality of semantic slots in the semantic slot template from the medical consultation sentence.

13. The medical question-answering system according to claim 12, wherein the recognition unit is further configured to:
perform sequence labeling on the medical consultation sentence by using a sequence labeling model, and acquire the substantive terms corresponding to the plurality of semantic slots in the semantic slot template according to a sequence labeling result.

14. The medical question-answering system according to claim 12, wherein the parser comprises:
a filling unit, configured to fill the standard term corresponding to the medical consultation sentence of the patient into corresponding one of the plurality of semantic slots;
a determining unit, configured to determine whether there is an unfilled semantic slot in the semantic slot template;
an inquiring unit, configured to, in a case where there is an unfilled semantic slot in the semantic slot template, generate an inquiry question corresponding to the unfilled semantic slot, and fill the unfilled semantic slot according to an answer sentence input by the patient for the inquiry question, until all of the semantic slots in a current semantic slot template are filled; and
a parsing unit, configured to generate the semantic parsing result according to the intention of the patient, each semantic slot, and a filled value of the semantic slot.

15. The medical question-answering system according to claim 10, wherein the output unit comprises:
a matching degree calculator, configured to calculate a matching degree between the semantic parsing result and each sample group in a doctor-patient question-answering knowledge library, wherein each sample group comprises a question sample and its corresponding answer sample; and
an output subunit, configured to output an answer sample having a maximum matching degree.

16. The medical question-answering system according to claim 15, wherein the matching degree calculator comprises:
a calculation subunit, configured to calculate a similarity between the semantic parsing result and the question sample and a correlation between the semantic parsing result and the answer sample; and
a generation subunit, configured to generate the matching degree according to the similarity, a first weighting coefficient, the correlation, and a second weighting coefficient.

17. The medical question-answering system according to claim 10, further comprising:
a standard term library generator, configured to generate a standard term library in which a plurality of standard term samples are stored;
a synonym collector, configured to collect at least one synonym corresponding to each of the plurality of standard term samples;
a filter, configured to calculate a similarity between each standard term sample and its corresponding synonym, maintain each synonym having a similarity greater than a preset value, and remove each synonym having a similarity less than or equal to the preset value; and
a mapping table generator, configured to generate the synonym mapping table according to each standard term sample and its corresponding synonym which is maintained currently.

18. An electronic device, comprising a processor and a memory which has a computer program stored thereon, wherein the computer program, when executed by the processor, implements the medical question-answering method according to claim 1.

19. A non-transitory computer readable storage medium on which a computer program is stored, wherein the computer program, when executed by a processor, implements the medical question-answering method according to claim 1.

20. The medical question-answering method according to claim 2, wherein the extracting at least one substantive term corresponding to disease characteristics from the medical consultation sentence according to the intention of the patient comprises:
acquiring a semantic slot template corresponding to the intention of the patient, wherein the semantic slot template comprises a plurality of semantic slots for representing the disease characteristics; and
extracting substantive terms corresponding to the plurality of semantic slots in the semantic slot template from the medical consultation sentence.
